(19) United States
(12) Patent Application Publication
(10) Pub. No.: US 2003/0113379 A1
(43) Pub. Date: Jun. 19, 2003

(54) Biodegradable composite matrix for enhancing angiogenesis and method for producing the same

(75) Inventors: Da-Young Chen, Taipei (TW); Huey-Min Lai, Hsinchu (TW); Yuan-Han Chen, Hsinchu (TW); Rung-Jiun Gau, Kaohsiung (TW)

Correspondence Address: SUGHRUE MION, PLLC, 2100 PENNSYLVANIA AVENUE, N.W., WASHINGTON, DC, 20037, US

(73) Assignee: INDUSTRIAL TECHNOLOGY RESEARCH INSTITUTE

(21) Appl. No.: 10/219,674

(22) Filed: Aug. 16, 2002

(30) Foreign Application Priority Data
Dec. 19, 2001 (TW) 90131491

(51) Int. Cl. A61K 31/66; A61K 9/14

(52) U.S. Cl. 424/486; 424/488; 514/114

(57) ABSTRACT

A biodegradable composite matrix for enhancing angiogenesis. The matrix includes a biodegradable material and a phospholipid distributed in the biodegradable material as a liposome or micelle. Derivatives of phospholipid have chemo-attraction properties and thus induce the transfer of endothelial cells into the system through tropism-inductive mechanism. The biodegradable composite matrix induces an intracellular signal cascade that is different from cascade induced by a growth factor, and can coordinate with the growth factor-induced cascade to enhance angiogenesis. The present invention also includes the method for producing the biodegradable composite matrix.

### BACKGROUND OF THE INVENTION

1. Field of the Invention

The present invention relates to an angiogenesis promoting matrix. The present invention particularly relates to a biodegradable composite matrix for enhancing angiogenesis and method for producing the same.

2. Description of the Related Art

When the body undergoes a pathological change or is injured, the progress of angiogenesis often indicates the stage of recovery. However, in some pathological states, for example, wounds caused by chronic disease (such as diabetes and venous disease) and coronary artery clogging, angiogenesis cannot be carried out by the body properly and thus hampers the recovery. Therefore, induction or enhancement of proper angiogenesis is a major focus of development in the field of medical technique, especially in the field of tissue engineering. The technique will play an important role in the treatment of many disorders.

Angiogenesis is a very complex process involving intercellular interaction, growth factor activity and intracellular matrix activity. Tissue releases angiogenic growth factors around the site of injury or illness. These angiogenic growth factors bind to receptors on endothelial cells to activate the cells, which in turn release specific enzymes and growth factors. These enzymes and growth factors decompose base membranes of the blood vessels to allow the migration of blood vessel endothelial cells to the injured location and thus induce the angiogenetic mechanism. The control of angiogenesis promotion or inhibition has significant potential in disease treatment. Angiogenic growth factors play an important role in controlling angiogenesis and therefore have drawn much attention from many research groups (both academic and industrial) to search for suitable angiogenic growth factors and drugs using many human and material resources. Producing angiogenic growth using in vitro tissue culture has been tried. Other research groups have also tried to enhance wound healing by applying angiogenic growth factors on wounds.

Blood vessel growth is an important factor in the development of organ regeneration technology. In order to provide enough blood supply to a tissue-cultured organ (such as pancreas, liver, or kidney), angiogenesis enhancement is necessary. Providing an appropriate material for angiogenesis to promote the blood vessel growth is an important goal in tissue culture engineering. So far, materials used in promoting angiogenesis usually compose chemical macromolecule polymers as the matrix for attachment of cells, and angiogenic growth factors are added to promote cell growth. Low bioacceptability is a problem when chemical macromolecule polymer is used as the matrix, and the slow diffusion rate of the added angiogenic growth factors is also to be considered. Undesired protein denaturalization and function loss often occur during manufacture of such material. Moreover, such material is difficult to preserve and is very expensive. Therefore the development of a natural biological macromolecule composite matrix with high bioacceptability, low cost, and high efficacy will be of great help in this field.

Some basic research has shown that, although the addition of growth factors stimulates cell division, promotes cell migration and vessel formation, it also has following disadvantages:

1. Large molecular weight decreases diffusion rate: Most growth factors (such as bFGF and VEGF) have molecular weight between 1-2 kDa. Such large molecular weight hampers proper diffusion throughout the tissues, and often fails to form a growth factor concentration gradient, which is desired. Therefore cell growth cannot be properly orientated.

2. The growth factors can be attacked by the immune system: growth factors often induce immune response in a foreign organism, and can lead to fatal allergic reaction.

3. Mutability: Most growth factors are composed of proteins with complex structure and molecule interaction. Denaturalization of growth factors can thus easily occur during changes of physical or chemical environment resulting in the loss of activity.

4. High cost: Growth factors are usually produced by gene recombination, and therefore with high cost.

### SUMMARY OF THE INVENTION

Therefore, the object of the present invention is to provide a biodegradable composite matrix for enhancing angiogenesis and method for producing the same.

In order to solve the previously mentioned problems, the present invention provides a biodegradable material containing phospholipid capable of promoting angiogenesis. Derivatives of phospholipid do not have the difficulty in diffusion growth factors do. Derivatives of phospholipid also have chemo-attraction properties and thus induce the transfer of endothelial cells into the system through tropism-inducing mechanism. The biodegradable composite matrix of the present invention induces an intracellular signal cascade that is different from cascade induced by a growth factor, and can coordinated with the growth factor-induced cascade to enhance angiogenesis. The present invention also includes the method for producing the biodegradable composite matrix. The advantages of the present invention are:

1. The addition of active phospholipid into the carrier promotes angiogenesis.

2. Active phospholipid in the form of liposome is added to a biodegradable material. The angiogenesis enhance system is easy to manufacture, and the system is stable.

3. The scope of application is widened since phospholipid is a dipole molecule and can integrate with both hydrophilic and hydrophobic materials.

4. Due to the dipole property and special critical micelle concentration (CMC) of phospholipid, continued release can be achieved. The release curve can be adjusted by manufacture or transformation of the matrix to meet different requests of different clinical situations.

### DETAILED DESCRIPTION OF THE PREFERRED EMBODIMENT

The biodegradable composite matrix for enhancing angiogenesis of the present invention comprises: (a) a biodegradable material, and (b) a phospholipid distributed in the biodegradable material as a liposome or micelle.

The phospholipid of the present invention is more stable than growth factors, and can stably exist alone. The phospholipid has a small molecular weight ranging from about 300-400 Da, and therefore diffuses faster throughout the tissue matrix. In fact, the phospholipid is released by activated platelets in the body. Its functions include attracting endothelial cell migration, stimulating cell growth, and preventing tissue fiberization by suppressing smooth muscle cell and fibroblast growth. Phospholipid is also present in cell membrane and plays an important role as a signal transducer. Most cellular signal transduction pathways and transducers are conserved in higher animals. Phospholipid does not have the problem of rejection due to its generality and small molecular weight.

Preferred phospholipid in the present invention includes, but is not limited to: sphingosine-1-phosphate (S-1-P), lysophosphatidic acid (L-P-A) or other phospholipids isolated from platelets or cell membrane, wherein S-1-P is more preferable. Since S-1-P is a dipole molecule with higher critical micelle concentration (CMC), it exists as a single micelle layer in the composite matrix and is capable of continuously releasing S-1-P monomers and diffuses into tissues rapidly. Therefore the composite matrix of the present invention continuously induces endothelial cell migration and promotes local blood vessel growth and wound healing. Moreover, the receptor to S-1-P, S-1-P receptor, is a G-protein-coupled receptor, which activates cells and transfers signals by activating various G-proteins. On the other hand, activation mechanism induced by protein growth factors is normally a receptor tyrosine kinase (RTK) pathway. These two pathways act independently but can effect each other to some extent. Therefore, the material of the present invention can contain a suitable growth factor apart from phospholipid when desired. In this way, the phospholipid derivative stimulates endothelial cell division, together with the activity of the growth factor, a better angiogenic effect can be achieved.

Preferred biodegradable material of the present invention includes, but is not limited to: gelatin, collagen, chitin, chitosan, cellulous and its derivatives, glucosaminoglycans, chondroitin sulfates, hyaluronic acid, or other extracellular matrix (ECM) proteins, alginates, starch/modified starch, carragenam/salts, pectins, other ECM polysaccharides, polylactide glycolide acid (PLGA), and other materials obtained from biological organisms. Natural macromolecule complex with advantages in medical application such as low toxicity, biodegradability, high bioacceptability is more preferable.

According to a preferred embodiment of the present invention, collagen and polysaccharide are used as the biodegradable material. The biodegradable material has dual cell receptor, special cell affinity ligand, dual enzymatic degradation pathway and degradation rate, and therefore the degradation period is extended and attachment of certain cells is allowed. Moreover, the collagen/polysaccharide complex material fuses with S-1-P micelle very well and allows S-1-P to evenly distribute in the material. Collagen/polysaccharide complex material contains various hydrophilic functional groups, which capture many water molecules and thus prevent dehydration during gel formation, which causes discharge of S-1-P and growth factor from the material.

The method of the present invention comprises the following steps: Dissolving phospholipid and spreading the solution on the inner wall of a container. Phospholipid micelle is then produced by phase separation with temperature differences and sonication. The phospholipid micelle is mixed with the biodegradable material to form the biodegradable composite matrix for enhancing angiogenesis of the present invention.

More detailed procedure of the method of the present invention is as follows: (a) phospholipid is dissolved in a solvent in a container; (b) the solvent is removed by decompression distillation, allowing the phospholipid to adhere to the inner wall of the container; (c) phosphate buffered saline is added (PBS), and frozen rapidly; (d) sonication is then carried out to allow the phospholipid to form micelle in PBS; (e) the phospholipid micelle is mixed with a biodegradable material to form a biodegradable composite matrix for enhancing angiogenesis.

The solvent used in step (a) is a polar organic solvent, and is often an alcohol such as methanol or ethanol. The freezing step in step (c) is preferably carried out using liquid nitrogen. In step (e), the biodegradable material can be manufactured into matrixes such as hydrogel, porous matrix, film matrix, fiber matrix, tubular matrix, creaming matrix prior to mixing with phospholipid micelle. Otherwise, phospholipid can first be mixed with degradable material and then manufactured into the previously mentioned matrixes. For example, S-1-P micelle is mixed with collagen solution (0.6-1.0 w/v in 1% organic acidic solvent with 1% acetic acid or lactic acid) and then freeze-dried to form porous composite matrix. Composite matrix in the hydrogel form can be introduced into a test body via subcutaneous injection, while other forms such as porous matrix, can be introduced into the derma or hypoderm via a surgical operation.

Protein growth factors, inhibitors or cytokines can also be added into the composite matrix of the present invention. Suitable growth factors include, but are not limited to: fibroblast growth factor (FGF), placental growth factor, transforming growth factor, angiogenin, interleukin-8, hepatocyte growth factor, hepatocyte growth factor, granulocyte colony-stimulating factor, and platelet-derived endothelial cell growth factor. Suitable inhibitors include, but are not limited to: interferon α, transforming growth factor β, thrombospondin-1, angiostatin, placental proliferin-related, platelet factor 4, genistein, metallo-proteinase inhibitor, and prolactin 16-kd fragment.

Accordingly, the biodegradable composite matrix for enhancing angiogenesis of the present invention has the following advantages:

1. The composite matrix has advantages in medical application such as low toxicity, biodegradability, and good bioacceptability.

2. The hydrogel system of the composite matrix has extracellular matrix-like structure, which is suitable for cell attachment and growth.

3. The composite matrix promotes angiogenesis.

4. The composite matrix is easy to produce and preserve, and thus damage to the carrier during manufacturing is reduced.

5. Phospholipid is a dipole molecule capable of fusing with hydrophilic or hydrophobic material and thus has a wide scope of application.

6. Other active factors (such as growth factors and drugs) can be added into the composite matrix to meet the requirement in applications such as drug release or tissue engineering.

Without intending to limit it in any manner, the present invention can be more fully understood by reading the subsequent examples and references made to the accompanying drawings.

### BRIEF DESCRIPTION OF THE DRAWINGS

FIG. 1 is a diagram showing result of mouse tissue sections stained with HE of Example 1, wherein (a) is treated with bFGF and S-1-P, (b) is treated with bFGF, and (c) is the control sample treated with PBS.

FIG. 2 is average blood vessel numbers of the tissue sections, wherein blood vessel number in the bFGF/S-1-P sample is larger than the bFGF sample, which is in turn larger than the PBS sample.

### EXAMPLE 1

### Hydrogel System Without Collagen

The composite matrix of the present example is a biodegradable natural macromolecule complex containing polysaccharide (chitin) and phospholipid S-1-P. It was produced as follows:

S-1-P was dissolved in methanol and the solution was poured into a round bottom flask. S-1-P was then dried and spread onto the inner wall of the flask by decompression distillation. PBS was added into the flask followed by rapid freezing using liquid nitrogen. The mixture was then sonicated under room temperature to melt the S-1-P from the inner wall and form micelles. The resulting S-1-P micelles were mixed with chitin hydrogel and bFGF to give a composite matrix. The composite matrix was introduced into the mouse via subcutaneous injection.

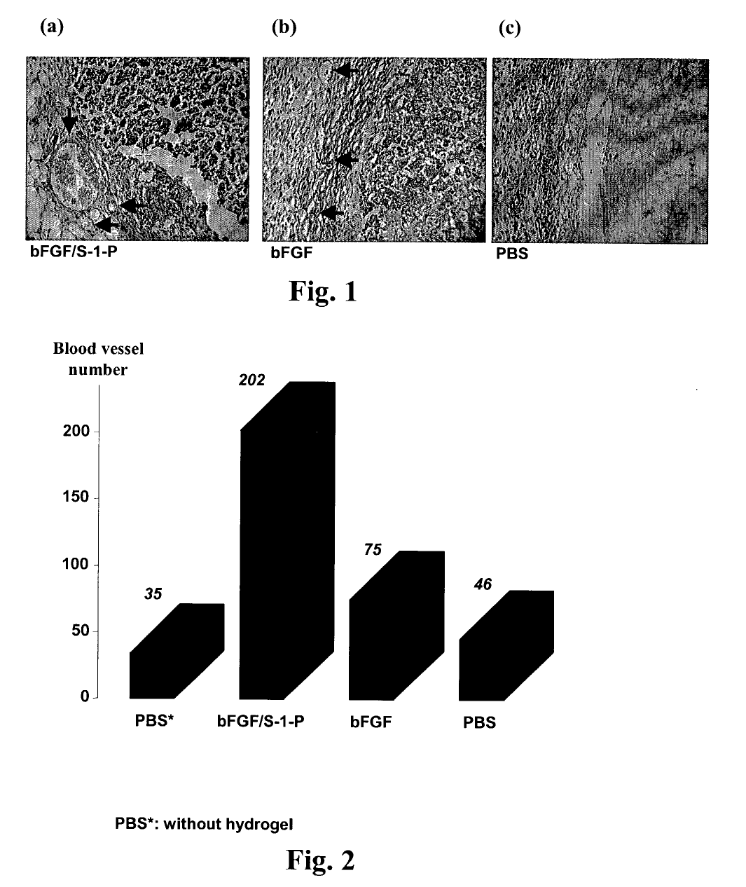

FIG. 1 shows the tissue sections of the mice stained with HE stain. The mice were sacrificed 15 days after injecting the hydrogel. The implanted tissues were obtained, fixed, embedded, sectioned, stained, and observed under a 200 times microscope. The sizes and conditions of the blood vessels of the sections were observed. FIG. 1(a) shows the tissue section from the mouse treated with both bFGF and S-1-P. It is shown that both diameter and number of blood vessel in this tissue section are obviously larger than those of FIG. 1(b), where only bFGF was used, and those of FIG. 1(c), which is the control. The arrows indicate the blood vessels. FIG. 2 is the average numbers of blood vessels of the tissue sections. It is shown that blood vessel number of the sections is bFGF/S-1-P>bFGF>PBS, which is in consistent with results shown in FIG. 1.

### EXAMPLE 2

### Hydrogel System With Collagen

The composite matrix of the present example is a biodegradable natural macromolecule complex containing protein (collagen), polysaccharide (chitin) and phospholipid S-1-P.

S-1-P was dissolved in methanol and the solution was poured into a round bottom flask. S-1-P was then dried and spread onto the inner wall of the flask by decompression distillation. PBS was added into the flask followed by rapid freezing using liquid nitrogen. The mixture was then sonicated under room temperature to melt the S-1-P from the inner wall and form micelles. The resulting S-1-P micelles were mixed with chitin-collagen heat-sensitive hydrogel to give a composite matrix containing collagen, chitin, and phospholipid S-1-P.

### EXAMPLE 3

### Porous Matrix

The composite matrix of the present example is a biodegradable natural macromolecule complex containing collagen porous sponge and phospholipid S-1-P.

S-1-P was dissolved in methanol and the solution was poured into a round bottom flask. S-1-P was then dried and spread onto the inner wall of the flask by decompression distillation. PBS was added into the flask followed by rapid freezing using liquid nitrogen. The mixture was then sonicated under room temperature to melt the S-1-P from the inner wall and form micelles. The resulting S-1-P micelles were mixed with collagen porous sponge to give a composite matrix.

While the invention has been described by way of example and in terms of the preferred embodiment, it is to be understood that the invention is not limited to the disclosed embodiments. On the contrary, it is intended to cover various modifications and similar arrangements as would be apparent to those skilled in the art. Therefore, the scope of the appended claims should be accorded the broadest interpretation so as to encompass all such modifications and similar arrangements.

What is claimed is:

1. A biodegradable composite matrix for enhancing angiogenesis comprising:
(a) a biodegradable material, and
(b) a phospholipid distributed in the biodegradable material as a micelle.

2. The biodegradable composite matrix as set forth in claim 1, wherein the biodegradable material is gelatin, collagen, chitin, chitosan, glucosaminoglycans, chondroitin sulfates, hyaluronic acid, alginates, starch/modified starch, carragenam/salts, pectins, polylactide glycolide acid (PLGA), or any combination thereof.

3. The biodegradable composite matrix as set forth in claim 1, wherein the biodegradable material is collagen/polysaccharide complex material.

4. The biodegradable composite matrix as set forth in claim 1, wherein the phospholipid is sphingosine-1-phosphate (S-1-P).

5. The biodegradable composite matrix as set forth in claim 1, wherein phospholipid is lysophosphatidic acid (L-P-A).

6. The biodegradable composite matrix as set forth in claim 1, wherein the phospholipid is isolated from platelet or cell membrane.

7. The biodegradable composite matrix as set forth in claim 1, wherein the biodegradable material is in the form of a hydrogel, porous matrix, film matrix, fiber matrix, tubular matrix, or creaming matrix.

8. The biodegradable composite matrix as set forth in claim 1, further comprising a growth factor, an inhibitor or a cytokine.

9. The biodegradable composite matrix as set forth in claim 8, wherein the growth factor is fibroblast growth factor (FGF), placental growth factor, transforming growth factor, angiogenin, an interleukin-8, hepatocyte growth factor, hepatocyte growth factor, granulocyte colony-stimulating factor, or platelet-derived endothelial cell growth factor.

10. The biodegradable composite matrix as set forth in claim 8, wherein the inhibitor is interferon α, transforming growth factor β, thrombospondin-1, angiostatin, placental proliferin-related, platelet factor 4, genistein, metallo-proteinase inhibitor, or prolactin 16-kd fragment.

11. A method for producing a biodegradable composite matrix for enhancing angiogenesis comprising the following steps:
(a) dissolving a phospholipid in a solvent in a container;
(b) removing the solvent by decompression distillation and allowing the phospholipid to attach to the inner wall of the flask;
(c) adding phosphate buffered saline and freezing the mixture rapidly;
(d) sonicating the mixture to allow the phospholipid to form micelles in PBS; and
(e) mixing the phospholipid micelles with a biodegradable material to form a biodegradable composite matrix for enhancing angiogenesis.

12. The method for producing a biodegradable composite matrix as set forth in claim 11, wherein the solvent is a polar organic solvent.

13. The method for producing a biodegradable composite matrix as set forth in claim 12, wherein the solvent is an alcohol.

14. The method for producing a biodegradable composite matrix as set forth in claim 11, wherein the phospholipid is sphingosine-1-phsophate, lysophosphatidic acid, or phospholipid isolated from platelet or cell membrane.

15. The method for producing a biodegradable composite matrix as set forth in claim 11, wherein step (c) is carried out using liquid nitrogen.

16. A method for producing a biodegradable composite matrix as set forth in claim 11, wherein the biodegradable material is gelatin, collagen, chitin, chitosan, cellulous and its derivatives, glucosaminoglycans, chondroitin sulfates, hyaluronic acid, alginates, starch/modified starch, carragenam/salts, pectins, polylactide glycolide acid (PLGA), or any combination thereof.

17. The method for producing a biodegradable composite matrix as set forth in claim 11, wherein the biodegradable material is a collagen/polysaccharide complex material.

18. A method for producing a biodegradable composite matrix as set forth in claim 11, wherein the biodegradable material is in the form of a hydrogel, porous matrix, film matrix, fiber matrix, tubular matrix, or creaming matrix.

19. The method for producing a biodegradable composite matrix as set forth in claim 11, wherein step (e) further comprises adding a growth factor, an inhibitor, or a cytokine into the biodegradable material.

20. The method for producing a biodegradable composite matrix as set forth in claim 19, wherein the growth factor is fibroblast growth factor (FGF), placental growth factor, transforming growth factor, angiogenin, an interleukin-8, hepatocyte growth factor, hepatocyte growth factor, granulocyte colony-stimulating factor, or platelet-derived endothelial cell growth factor.

21. A method for producing a biodegradable composite matrix as set forth in claim 19, wherein the inhibitor is interferon α, transforming growth factor β, thrombospondin-1, angiostatin, placental proliferin-related, platelet factor 4, genistein, metallo-proteinase inhibitor, or prolactin 16-kd fragment.
